(19) United States
(12) Patent Application Publication
(10) Pub. No.: US 2023/0414245 A1
(43) Pub. Date: Dec. 28, 2023

(54) Catheter Assembly

(71) Applicant: Ureca B.V., Rotterdam (NL)

(72) Inventors: Johannes Franciscus Marinus Remmerswaal, Den Hoorn (NL); Jan Willem Kuiper, Rotterdam (NL)

(73) Assignee: Ureca B.V., Rotterdam (NL)

(21) Appl. No.: 18/253,937

(22) PCT Filed: Nov. 24, 2021
(86) PCT No.: PCT/EP2021/082870

§ 371 Date: May 23, 2023

(30) Foreign Application Priority Data
Nov. 25, 2020 (NL) 2026982

(51) Int. Cl. A61B 17/3207

(52) U.S. Cl. CPC A61B 17/3207; A61B 2017/00336

(57) ABSTRACT

Catheter assembly for passing an occlusion in a vessel, having a catheter hub; a hollow flexible shaft extending between the catheter hub and an open distal end; and a cutting wire provided inside the shaft, the cutting wire extending from the catheter hub to the distal end. The shaft has a cannula lumen with a cannula opening at the distal end, the cannula lumen being configured to guide a cannula. The catheter hub has a handle, which is configured to operate selectively in a cutting wire mode and in a cannula mode, and a blocking mechanism, configured to block the cannula from extending from the cannula opening while the cutting wire sticks out from the distal end.

### BACKGROUND OF THE INVENTION

### 1. Field of the Invention

The present invention relates to a catheter assembly for passing an occlusion in a vessel. The invention also relates to a cannula for use in such a catheter assembly. Furthermore, the invention relates to a method of operating a catheter assembly.

### 2. Description of the Related Art

In order to pass occlusions in blood vessels such as veins or arteries, catheter assemblies with surgical cutting instruments may be used. Such assemblies may be introduced into the respective vessels and the occlusion passed by means of e.g. a cutting wire at the distal end of the instrument.

An example of such an instrument is known from patent application WO 2010/151119. In this document, the cutting instrument includes a cutting wire which penetrates the vessel wall to pass the occlusion and then re-enter the vessel distal to the occlusion.

Although this cutting instrument represents a significant improvement over the prior art, there are still situations in which re-entry into the vessel is unsuccessful or in which desired actions in the vessels cannot be performed.

In light of the above, it would be desirable to provide solutions which at least partially overcome some of the inconveniences of the prior art.

### SUMMARY OF THE INVENTION

According to the invention there is provided a catheter assembly for passing an occlusion in a vessel, comprising:
a catheter hub;
a hollow flexible shaft extending between the catheter hub and a distal end wherein the shaft comprises a cannula lumen with a cannula opening at the distal end, the cannula lumen being configured to guide a cannula, and at least one cutting wire lumen;
a cutting wire provided inside the shaft, the cutting wire extending through the cutting wire lumen from a cutting wire carrier within the catheter hub to a cutting wire opening at the distal end;
a cannula carrier, provided within the catheter hub, the cannula carrier being configured to receive a cannula and being aligned with the cannula lumen whereby a cannula can pass through the cannula carrier and through the cannula lumen;
   wherein the cannula carrier and the cutting wire carrier are slideably received within the catheter hub to allow extension and retraction of the respective cannula and cutting wire and the catheter hub is configured to operate selectively in a cutting wire mode and in a cannula mode, wherein
in cutting wire mode a user can act on the cutting wire carrier to move the cutting wire to extend from the cutting wire opening and to be retracted into the shaft;
in cannula mode the user can act on the cannula carrier to move the cannula to extend from the cannula opening; and
   wherein the catheter hub further comprises a blocking mechanism, configured to block movement of the cannula carrier while the cutting wire extends from the cutting wire opening.

Although reference is given to a cannula, it should nevertheless be understood that the catheter assembly may be delivered and even used without a cannula and/or that other elements may be placed into the cannula carrier and cannula lumen, such as a micro-catheter, stylet, guidewire, dilatation device, optical fibre, endoscope or the like. In a preferred embodiment, the catheter assembly further comprises a cannula for placement inside the cannula lumen, configured to extend between the catheter hub and the distal end. The cannula extends from a proximal cannula end to a distal cannula end and has a hollow core.

Such a catheter assembly then combines the functions of a cutting wire with the functions of a cannula. In other words, the catheter assembly allows the user/operator to cut through tissue at the vessel wall to leave the vessel, and to subsequently re-enter into the vessel, for instance after passing an occlusion. The cannula is formed as an elongate, flexible member which may be made of a polymer, such as PEEK or metal, such as nitinol. The cannula and the shaft may be made of the same material. The flexible member extends from a cannula hub for fixing it to the catheter hub to a cannula distal end, which may be sharpened. The cannula is preferably less flexible but sharper than the cutting wire, such that it is more suited to cut through subluminal tissue and/or calcified tissue to force re-entry into the vessel. The cannula may thus be used as a needle. The cannula may be configured to place a guide wire into the vessel and/or to deliver a substance (e.g. contrast liquid) into the vessel, via a hollow core in the cannula. In this way, a user can simultaneously perform the PIER (percutaneous intentional extraluminal recanalization) method and re-enter into the vessel using the same device. The cutting wire and cannula preferably operate along the length of the shaft and are both configured to protrude from the distal end of the shaft, such that they operate in line with one another. The cutting wire and cannula are preferably employed successively.

The catheter assembly according to the invention has the advantage that the cutting wire and cannula cannot be extended simultaneously. The blocking mechanism ensures that the cannula can only be employed when the cutting wire does not extend from the shaft, i.e. is fully retracted into or onto the shaft. The risk of interference between cutting wire and cannula inside the vessel is thus avoided. By full or complete retraction is meant that the cutting wire is accommodated inside the shaft, such that it becomes impossible for the cannula to interfere with the cutting wire when it extends from the distal end of the shaft. The cutting wire preferably comprises a loop at its distal end, connecting the ends of two portions of cutting wire which extend in parallel through the shaft, each in their own cutting wire lumen extending to a cutting wire opening. If that is the case, full retraction may mean that the loop is shrunk to a negligible size whereby the cutting wire extends over a minimum distance between the cutting wire lumen outlets. It is thus preferred that the catheter hub can only switch from cutting wire mode to cannula mode when the cutting wire is fully retracted. Moreover, when the catheter is removed from the vessel, the cutting wire is preferably fully retracted inside the shaft such that it cannot interact with e.g. a stent located proximally in the vessel. In particular if the cutting wire comprises a loop, this forms a significant risk which is avoided if the cutting wire is fully retracted.

As an alternative to a loop, the cutting wire may comprise a bent end such that the sharp point of the cutting wire directs sideways or backwards with respect to the shaft's longitudinal axis. In this way it is ensured that the sharp point does not directly engage with tissue to avoid damage.

The distal end of the shaft preferably comprises multiple distinct openings, associated with either the cutting wire or the cannula. In the case that the cutting wire forms a loop, there may be two cutting wire lumens and two cutting wire openings. Alternatively, the cutting wire may be attached close to the shaft's distal end, and form a loop when the cutting wire extends further from the cutting wire opening. This is depicted in FIGS. 1-2 of WO 2009/082228, which is incorporated herein by reference in its entirety. A person skilled in the art will understand that also the other alternatively formed loops disclosed in WO 2009/082228 are within the scope of this embodiment.

The catheter hub may be blocked from switching back from cannula mode to cutting wire mode in order to avoid interference between cannula and cutting wire after employing the cannula and in order to ensure that the cutting wire is fully retracted during removal of the catheter.

In one embodiment, the catheter hub includes at least one handle and the handle is configured to allow selection of the cutting wire mode and cannula mode, wherein
in cutting wire mode the handle operatively engages with the cutting wire carrier such that a user can act on the handle to move the cutting wire to extend from the cutting wire opening and to be retracted into the shaft;
in cannula mode the handle operatively engages with the cannula carrier such that a user can act on the handle to move the cannula to extend from the cannula opening.

The handle is preferably movable with respect to the catheter hub. Being ‘movable’ refers to sliding, tilting, shifting, rolling (such as wheels) or the like. Nevertheless, electronic actuation is also contemplated and buttons or switches are also considered to be handles in the context of the broadest aspect of this invention.

As an alternative to a single handle which can switch between cutting wire mode and cannula mode, the catheter hub may include two handles, i.e. a first handle and a second handle, wherein the first handle is connected to or is configured to operatively engage with the cutting wire carrier such that a user can act on the first handle to move the cutting wire to extend outside from the cutting wire opening and to be retracted into the shaft; and wherein the second handle is connected to or configured to operatively engage with the cannula carrier such that a user can act on the second handle to move the cannula to extend from the cannula opening. In this version, which may be combined with other preferred features as described herein, the second handle is configured to only operate while the cutting wire is fully retracted. The advantages of a single handle (which can selectively operate in a cutting wire mode and a cannula mode) may also relate to two handles (one for controlling the cutting wire and one for controlling the cannula) which cannot operate simultaneously.

The catheter assembly may be used to pass occlusions in blood vessels, in a way that is similar to that described in WO 2010/151119, which is incorporated herein in its entirety by reference.

Although the cannula is a useful tool for forcing re-entry into the vessel, in certain circumstances using a cannula may not be required, since employing the cutting wire may already be sufficient to re-enter the vessel. In that case, a user may not utilise a cannula in the catheter assembly but, instead of placing a cannula inside the cannula lumen, directly place a guide wire in the cannula lumen, for instance a guide wire with a diameter of 0.89 mm (0.035 inch). If a user wishes to place a guide wire in a vessel, she/he thus has two options with the present catheter assembly: either using a cannula first and subsequently placing a thin guide wire, e.g., with a diameter of 0.46 mm (0.018 inch) or 0.36 mm (0.014 inch), within the cannula's hollow core, or directly placing a (thin or thicker) guide wire through the cannula lumen.

The catheter shaft preferably has a diameter of between 1 mm (3 French) and 3 mm (9 French), preferably about 2 mm (6 French). The shaft may alternatively have a diameter of about 1.3 mm (4 French). The shaft may have a length of between 20 cm and 150 cm; in an embodiment this length may be between 40 cm and 80 cm, preferably about 65 cm. In other embodiments, the shaft may be longer, for instance between 100 cm and 125 cm, preferably about 115 cm.

The cutting wire may be made of any appropriate material or combinations of materials, such as polyimide or nitinol and preferably has a diameter of between 0.1 mm and 1.5 mm, more preferably about 0.46 mm (0.018 inch). The part of the cutting wire that is to extend beyond the distal end of the catheter shaft is preferably between 25 mm and 100 mm long, preferably about 50 mm. If the cutting wire comprises a loop, these lengths refer to the maximum distance between the cutting wire opening and the distal extent of the loop.

The cannula lumen preferably has a diameter of between 0.4 mm and 1.3 mm, more preferably about 1 mm, such that it can accommodate a guide wire with a diameter of 0.89 mm (0.035 inch), or a cannula with an elongate, flexible member having a similar diameter. The cannula's elongate member may thus have a diameter between 0.35 mm and about 1.2 mm, preferably between 0.8 mm and 0.9 mm. The length of the cannula preferably corresponds to the length of the shaft and the catheter hub together, which may be between 50 cm to 170 cm, preferably about 90 cm or about 140 cm, depending on the shaft length. The elongate member of the cannula has a hollow core, which may have a diameter of between 0.2 mm and 0.6 mm, preferably about 0.4 mm, such that it can accommodate a guide wire with a diameter of 0.36 mm (0.014 inch).

The shaft may be of solid cross-section with the lumens inside being the only hollow parts of the shaft. Alternatively, the shaft may be a hollow tube-like component with an inner wall, wherein the lumens are themselves tubular components, e.g. glued or adhered to the inner wall of the shaft.

In an embodiment, the catheter hub further comprises an interlock button, preferably near to a proximal or first end of the catheter hub, wherein full retraction of the cutting wire into the shaft is only possible while the interlock button is actuated. This has the advantage that switching from cutting wire mode to cannula mode can only be done deliberately, since it requires actuation of the interlock button or, in the case of a handle, simultaneous actuation of the handle and of the interlock button. In practice, it may require both hands of a user to act simultaneously, while normal operation requires only a single hand.

In an embodiment, the handle is a slider and the catheter hub comprises a first slider channel and a second slider channel, which are preferably both oriented in a direction defined by a longitudinal axis of the shaft, and the slider is configured to slide through the first slider channel to control the advancement of the cutting wire in cutting wire mode; and the slider is configured to slide through the second slider channel to control the advancement of the cannula in cannula mode; and the first slider channel and second slider channel are connected such that the slider can move from the first slider channel to the second slider channel.

The first slider channel preferably has a length of between 25 and 100 mm, preferably between 40 and 75 mm. The second slider channel preferably has a length of between 3 and 20 mm, preferably between 4 and 8 mm. The lengths of the first and second slider channels may correspond to the lengths of the parts of the cutting wire and cannula that are to be extended outside from the distal end.

In an embodiment, the first slider channel and/or the second slider channel is provided with a toothed rack and the slider has at least one tooth for engaging with the toothed rack to block movement in a direction along the first or second slider channel, such that the slider can only slide through the first or second slider channel when the slider is depressed by a user to disengage the tooth and the rack.

In an embodiment, a first position of the handle corresponds to a fully retracted cutting wire, and the blocking mechanism comprises a safety feature configured to fix the handle at the first position when it reaches this position, such that the handle is blocked from advancing the cutting wire. In particular, the safety feature may be a safety detent to prevent the slider from moving through the first slider channel once it reaches the first position, such that the cutting wire cannot be advanced when the slider is in the first position. In this way, a user is able to deliberately fix the position of the handle at the fully retracted position of the cutting wire. This ensures that the catheter can safely be removed from the vessel without the vessel being exposed to a dragging cutting wire. At the same time, it ensures that the cannula can be extended without interfering with the cutting wire. The safety detent may be embodied by a resilient protrusion which can be passed by the handle in a first direction but which blocks passage of the handle in a second, opposite direction. The safety detent may have a tapered shape, narrowing towards the first position. Alternatively, the safety detent may be a removable blockade which blocks the handle, or another type of mechanical lock.

In an embodiment, the cannula has a cannula distal end and a cannula proximal end and the cannula comprises a cannula hub at the cannula proximal end. The cannula distal end may be configured to be inserted via the cannula carrier into the cannula lumen of the shaft and the cannula hub may be configured to be fixed e.g. irreversibly to the catheter hub or to the cannula carrier, preferably when the handle switches to cannula mode.

In an embodiment, the catheter hub is elongate and extends between a first end and an opposite second end, which is connected to the shaft. The catheter hub may comprise a grip for gripping the catheter hub by a user and the handle may be configured to move within a handle region which is between the grip and the second end, such that the movement of the handle can be controlled by a user's thumb.

As discussed above, the cutting wire may comprise a loop at its distal end. In an embodiment, the cutting wire is a single wire. Alternatively, the cutting wire includes two parallel cutting wire portions extending from the catheter hub to the distal end, which are continuous with one another at the distal end, so as to form the loop. Preferably, the two cutting wire portions are inside separate cutting wire lumens, their ends extending from cutting wire openings at the distal end, and wherein the ends are connected to one another outside the cutting wire lumens.

The loop forms a flexible and resilient cutting tool with a limited sharpness. The rigidity of the loop can be changed by enlarging or reducing the loop size. The loop may be formed by two parallel portions of cutting wire extending in cutting wire lumens all throughout the shaft.

Alternatively, the loop may be formed by one end of the cutting wire being fixed to the shaft, preferably near the distal end of the shaft. The location of the fixture of the cutting wire end to the shaft may be at the inside of the shaft, from which the cutting wire extends outwardly, forming a loop and returning into the shaft to the catheter hub. The location of the fixture of the cutting wire end to the shaft may alternatively be at the outside of the shaft, such that the cutting wire forms a loop before returning into the shaft to the catheter hub. This version of the loop is depicted in FIG. 4 of WO 2009/082228, which is incorporated herein in its entirety by reference.

When the cutting wire comprises a loop, the length of the cutting wire (as referred to above) corresponds to the maximum distance between a part of the loop and the distal end of the shaft.

The total length of the cutting wire that is extending from the distal end of the shaft is in that case two times the length of the loop.

As an alternative to the embodiment described in the previous paragraph, one end of the cutting wire may be fixed to the inside of the handle portion instead of in or on the shaft.

In an embodiment, the shaft comprises PEEK or is made of PEEK. PEEK is a high-performance thermoplastic material which meets the high mechanical and chemical resistance requirements of the invention. Alternatively, the shaft may comprise polyimide, polyamide, nylon or nitinol or is made of polyimide, polyamide, nylon or nitinol. The shaft may be a hypotube, which is an elongate tube, preferably made of metal, with micro-engineered features or patterns along its length, for instance to modify the mechanical properties of the tube.

In an embodiment, the shaft is braided to increase its torsional stiffness. Preferably, the distal end of the shaft responds directly to a rotation of the catheter hub to avoid any torsion forces built up in the shaft which may result in a sudden rotation of the distal end. Braiding is particularly advantageous for longer shafts, e.g. shafts over 100 cm.

The shaft may have a circular cross-section. Alternatively, the shaft may have an oval cross-section with an ovality (ratio of largest and smallest diameters) between 1 and 1.3, preferably between 1.05 and 1.2. If the shaft has an oval cross-section, the smallest diameter is preferably oriented parallel to a plane through the cutting wire lumen (if there are two of these). Advantageous to a shaft with an oval cross-section is the increased torsional stiffness. Braiding the shaft may further increase the torsional stiffness, yet has as a drawback that the flexibility of the shaft is reduced. In embodiments, the shaft may only have an oval cross-section or have an oval cross-section and be braided.

In an embodiment, the cutting wire and cannula are configured to extend from the distal end of the shaft in substantially the same direction, preferably parallel to an axis of symmetry of the distal end of the shaft. A small part at the very distal end of the shaft may have a negligible flexibility, when it is small enough, and defines the axis of symmetry.

According to another aspect of the invention, and in accordance with the effects and advantages described hereinabove, there is provided a cannula for use in a catheter assembly according to any one of the preceding claims, wherein the cannula comprises an elongate, flexible member and preferably a cannula hub for fixing the cannula to the catheter hub, the cannula hub being connected to one end of the flexible member. The flexible member is adapted for accommodation inside the shaft of the catheter assembly. The length of the flexible member corresponds to the combined length of the shaft and the catheter hub, such that the cannula can extend between the catheter hub and the distal end of the shaft. The flexible member is preferably made of PEEK, which is a material meeting the requirements in terms of hardness, flexibility and others. Moreover, it is less costly than nitinol, which is commonly used for these cannulas. The flexible member is conventionally hollow in order to accommodate contrast liquid and/or a guide wire, but a solid stylet of PEEK could also be used.

The cannula may alternatively be used without the catheter assembly and inserted directly into a patient's vessel. For instance, the cannula may be used for making a transjugular intrahepatic portosystemic shunt (TIPS). Such a shunt connects the portal vein which brings blood from the gastrointestinal tract and intra-abdominal organs to the liver via a transhepatic route (through liver tissue) via the right hepatic vein to the inferior vena cava to the right part of the heart. The procedure is carried out using fluoroscopy and ultrasound for guidance. The interventional radiologist punctures the jugular vein on the right side of the neck with the cannula and will then insert a vascular sheath over a wire passing the heart into the inferior vena cava. The right hepatic vein will be explored with a catheter designed for this purpose. The portal vein will be punctured through the liver with the cannula and a wire will be positioned between the two veins. After the area has been dilated, a stent or stent graft will be placed between the portal and hepatic vein to create a lasting connection.

The invention thus also relates to a method for making a transjugular intrahepatic portosystemic shunt (TIPS), including inserting a PEEK cannula as described herein into a vessel.

The cannula may also be used for radiofrequency (RF) ablation. When employing this method, the cannula is inserted into a patient's vessel (either directly or using a catheter assembly), and the tip of the cannula locally heats the tissue by means of an RF alternating current. When employed without catheter assembly, parts of the cannula are preferably isolated to ensure only the tissue around the cannula's tip is heated.

According to another aspect of the invention, and in accordance with the effects and advantages described hereinabove, there is provided a method for operating a catheter assembly as described herein, comprising:
positioning a cannula within the cannula lumen;
extending the cutting wire from the distal end;
fully retracting the cutting wire into the shaft; and subsequently
extending the cannula from the cannula opening.

In an embodiment, the method is used for passing an occlusion in a vessel, comprising:
introducing a shaft of a catheter assembly as described herein into the vessel;
wherein extending the cutting wire includes cutting through tissue of the vessel's wall, in order to pass the occlusion;
and wherein extending the cannula includes cutting through the tissue, in order to re-enter the vessel. In this context cutting through tissue of the vessel's wall means cutting through at least one layer of the tunica intima, tunica media or tunica adventitia of the vessel's wall.

In an embodiment, the method further comprises switching the handle from cutting wire mode to cannula mode after retracting the cutting wire.

The cannula may be placed into the catheter assembly at any point in time. This may happen before or after insertion of the shaft into the vessel. It may happen before or after the cutting wire has been employed, or even after full retraction of the cutting wire into the shaft.

Placing the cannula may only be possible when the cutting wire is fully retracted into the shaft, so either before or after employing the cutting wire.

In an embodiment, the method further comprises inserting a guide wire into and through the cannula to extend from the cannula distal end, distal of the occlusion; and retracting the catheter and cannula from the vessel while leaving the guide wire in position, across the conclusion.

### BRIEF DESCRIPTION OF THE DRAWINGS

The features and advantages of the invention will be further appreciated upon reference to the following schematic drawings of a number of exemplary embodiments, in which corresponding reference symbols indicate corresponding parts.

FIG. 1A-1C show part of a catheter assembly in cutting wire mode according to a first embodiment.

FIG. 1D shows a cross-section of the shaft of the catheter assembly in the direction I-D of FIG. 1B.

FIG. 1E shows a cross-section of a shaft of a catheter assembly according to an alternative embodiment.

FIG. 1F shows a cross-section of a shaft of a catheter assembly according to yet another embodiment.

FIGS. 2A-2B show the catheter hub of the catheter assembly of FIG. 1A in the transition from cutting wire mode to cannula mode.

FIG. 2C shows a detail of the distal end of the shaft of the catheter assembly of FIG. 1A;

FIGS. 3A-3B show the working of a handle of the catheter assembly of FIG. 1A;

FIGS. 4A-4D show the catheter assembly of FIG. 1A in cannula mode;

FIG. 5 shows an exploded view of the catheter assembly of FIG. 1A.

The figures are for illustrative purposes only, and do not serve as a restriction on the scope or the protection as laid down by the claims.

### DESCRIPTION OF ILLUSTRATIVE EMBODIMENTS

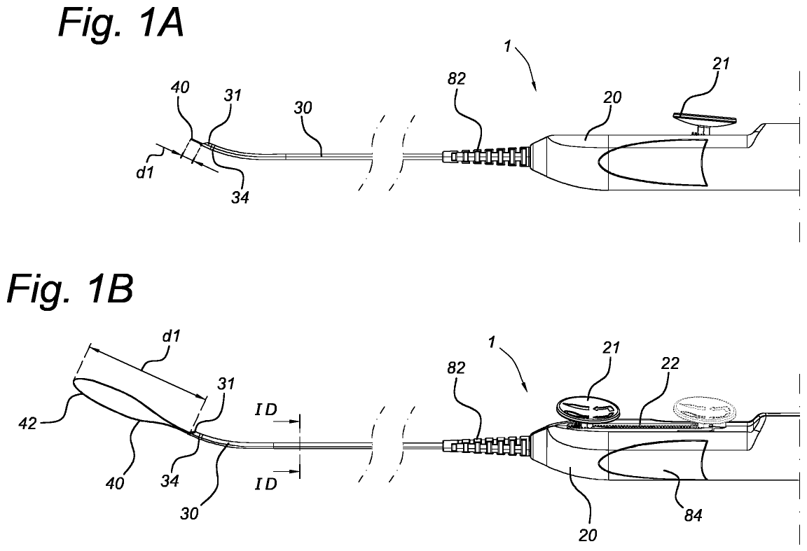

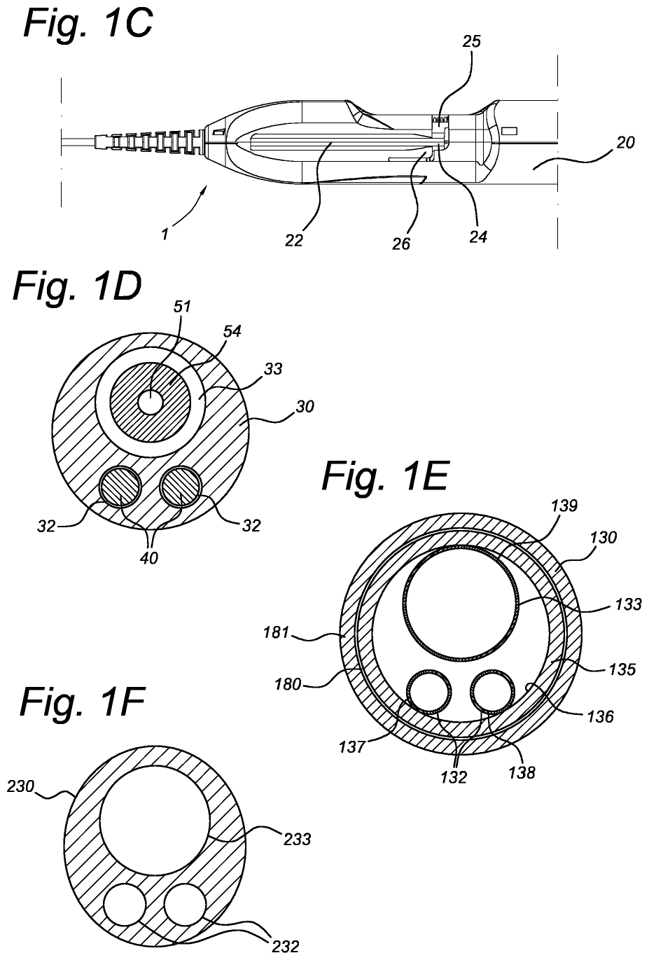

FIG. 1A schematically shows part of a catheter assembly 1 according to an embodiment of the invention. The catheter assembly 1 comprises a catheter hub 20, for instance made of plastic by injection-moulding, and a sleeve-like shaft 30 which is flexible and may be made of PEEK (polyetheretherketon) or other suitable materials. The shaft 30 may have a size of about 6 French (2 mm outer diameter), but it may be more or less (particularly between 3 and 9 French) depending on the requirements of the catheter assembly 1. A part of the length of the shaft is omitted in the drawing, as indicated by the curved lines. The catheter hub 20 has a first or proximal end that is omitted in the drawing and a second or distal end, which is connected to the shaft 30. A strain relief collar 82 is provided around the shaft 30 near the second end of the catheter hub 20, to protect the shaft 30 from damage or breaking near the catheter hub 20. A marker band 34 is fixedly provided at the distal end 31 of the shaft 30 which is radio opaque or visible with a desired imaging system, such as X-ray or MRI. The marker band 34 may be made of a metal such as platinum, iridium, stainless steel or gold, or made of tungsten-filled polymer or the like.

The catheter assembly 1 is depicted in cutting wire mode. A cutting wire 40 extends through the shaft 30 and extends for a first distance d1 from a cutting wire opening at the distal end 31 of the shaft 30. The length of the extended part of the cutting wire 40 is controlled by a handle, in this case a slider 21, which is mounted on the catheter hub 20 and which is internally connected to the cutting wire 40. The shaft 30 is bent near the distal end 31 to allow for careful positioning inside a vessel, and to direct the cutting wire 40 towards a vessel's wall. The bending of the shaft 30 is preferably in an upward direction with respect to the slider 21 in cutting wire mode. It will be understood that the bend in the shaft may be optional and that alternative shapes of shaft or even malleable shafts may be contemplated.

FIG. 1B schematically shows the same catheter assembly 1 of FIG. 1A in cutting wire mode, but now tilted slightly to show a first slider channel 22 through which the slider 21 may move in cutting wire mode. Below the first slider channel 22, a grip 84 is provided on the catheter hub 20 that allows the user to comfortably grip the catheter hub 20 and control the movement of the slider 21 using his/her thumb. The grip 84 is arranged at an end of the first slider channel and the slider 21 is configured to move in the first slider channel 22 between the grip 84 and the second end of the catheter hub 20. In FIG. 1A, the slider 21 is positioned in the most backward position in the first slider channel 22. In FIG. 1B, the slider 21 has been moved forward through the first slider channel 22 to increase the first distance d1 that the cutting wire 40 extends from the cutting wire opening. The part of the cutting wire 40 extending from the shaft 30 defines a loop 42, which is formed by the cutting wire 40 having a curve and extending back into the shaft 30 towards the catheter hub 20. In other words, the cutting wire 40 comprises two parallel cutting wire portions in the shaft 30 and the distal ends of the cutting wire portions are continuous with one another to form the loop 42. Inside the catheter hub 20, both proximal ends of the cutting wire 40 are operatively connected to the slider 21 in cutting wire mode, such that their position is controlled by the slider 21. In this way the size or length of the loop 42 is controlled too.

FIG. 1C shows a top view of a detail of the catheter assembly 1, but rotated and without the slider 21 for the ease of viewing. In particular, FIG. 1C shows a catheter hub 20 with the first slider channel 22. The slider 21 can be moved back and forth through the first slider channel 22 to control the first distance d1. The first distance d1 may be maximised to 25 mm to 100 mm, preferably about 50 mm. Only once the slider 21 has been moved out of the first slider channel 22 and into a first position 24 in a connection channel 25 with the cutting wire 40 fully retracted, can a switch to cannula mode be made. In order to avoid protruding the cutting wire 40 after it has been fully retracted, the first slider channel 22 is provided with a safety detent 26 which blocks forward movement of the slider 21 when it is located in the first position 24 of the connection channel 25. The safety detent 26 thereby blocks the slider 21 from re-entering the first slider channel 22 when the slider 21 has been positioned in the connection channel 25.

FIG. 1D schematically shows a cross-section of the shaft 30, indicated by ‘I-D’ in FIG. 1B. The shaft 30 comprises two cutting wire lumens 32 for guiding the cutting wire 40 through the shaft 30 between the shaft's distal end 31 and the catheter hub 20. The shaft 30 further comprises a cannula lumen 33, separate from the cutting wire lumen 32, for guiding a guide wire and/or a cannula. In FIG. 1D a hollow flexible elongate member 54 of a cannula is shown, which defines a hollow core 51 for accommodating a thin guide wire for placement into the vessel, or for delivering a substance into the vessel. Alternatively, the cannula may be removed and a guide wire may be inserted directly through the cannula lumen 33.

FIG. 1E schematically shows a cross-section of an alternative embodiment of a shaft 130. The shaft 130 also comprises two cutting wire lumens 132 and a cannula lumen 133. A cutting wire and cannula are not provided, yet the two cutting wire lumens 132 and cannula lumen 133 are configured to receive a cutting wire and cannula respectively. The shaft 130 is formed by a hollow tubular structure 135 with an inner wall 136. The cutting wire lumens 132 and cannula lumen 133 are formed by hollow tubes 137, 138, 139, which are adhered to the inner wall 136. In an embodiment, the hollow tubes 137, 138, 139 may be dimensioned such that they touch one another centrally in the tubular structure 135. Braid reinforcement 180 is provided around the tubular structure 135, which may be covered by a protection layer 181. The braid reinforcement 180 is here shown in combination with the hollow tubes 137, 138, 139, but it will be understood that the braid reinforcement 180 may equivalently be applied in the context of the embodiment shown in FIGS. 1A-1D.

FIG. 1F schematically shows a cross-section of yet an alternative embodiment of a shaft 230. The shaft 230 also comprises two cutting wire lumens 232 and a cannula lumen 233. A cutting wire and cannula are not provided, but the two cutting wire lumens 232 and cannula lumen 233 are configured to receive a cutting wire and cannula respectively.

The shaft 230 has an oval shape with an ovality of approximately 1.1. The shaft 230 has a largest diameter along a major axis of approximately 1.9 mm. The smallest diameter of the shaft is approximately 1.72 mm along a minor axis of the oval shaft. The minor axis intersects the cannula lumen 233 and the two cutting wire lumens 232 are arranged in a plane that is parallel to the minor axis.

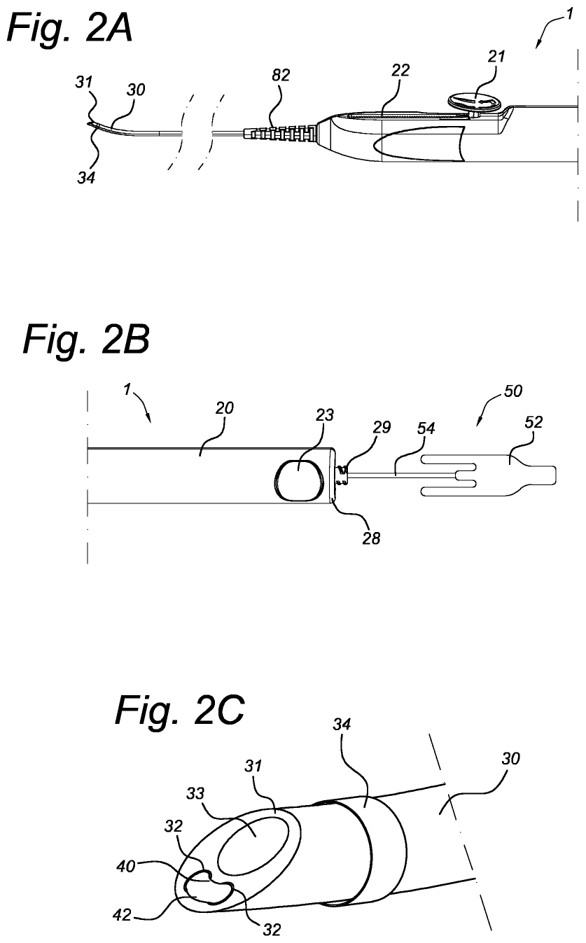

FIGS. 2A and 2B schematically show the catheter assembly 1 of FIGS. 1A-1D in fully retracted position, wherein the slider 21 is in the first position 24 in the connection channel 25 such that the cutting wire 40 is fully retracted inside the shaft 30. The catheter hub 20 comprises an interlock button 23. The interlock button 23 must be activated to fully retract the cutting wire 40 and move the slider 21 to this position. Without activation of the interlock button 23, the cutting wire 40 cannot be retracted further than the position as shown in FIG. 1A.

In the fully retracted position, a cannula 50 may be arranged in the catheter assembly 1. At the first and proximal end 28 of the catheter hub 20, a cannula carrier 29 is provided for receiving a schematically indicated cannula hub 52 with a flexible elongate member 54 extending from the cannula hub 52. The cannula carrier 29 comprises an entry channel and the flexible elongate member 54 is arranged to extend through the cannula carrier 29 into the catheter hub 20 and into the shaft 30. Although the catheter assembly 1 is configured to receive a cannula 50, the cannula 50 itself is not necessarily provided together with the catheter assembly 1 and is only shown here for explanatory purposes. As explained above, instead of the flexible elongate member 54 of the cannula 50 also a guide wire may be arranged in the cannula lumen 33. The cannula hub 52 is configured to be introduced into the catheter hub 20 at the first end 28. Upon insertion of the cannula hub 52, the cannula hub 52 and the cannula carrier 29 will engage with each other and interlock. This interlocking is not reversible, which means that the cannula 50 is no longer removable after complete introduction of the cannula hub 52 into the catheter assembly 1. Preferably, the cannula hub 52 is introduced into the catheter assembly 1 before the interlock button 23 is activated.

The interlock button 23 is an actuator of a blocking mechanism to be described in further detail below. Importantly, when it is intended that a cannula 50 extends from the cannula lumen 33, the blocking mechanism makes it impossible for the cannula 50 to interfere with the loop 42 of the cutting wire 40 by ensuring that the latter is fully retracted.

FIG. 2C schematically shows a perspective view of the distal end 31 of the shaft 30 when the cutting wire 40 is fully retracted inside the cutting wire lumens 32 as shown in FIG. 2A. As shown in this view, the distal end 31 of the shaft 30 is oblique, having a slanted face. It will be understood that other angles, including 90 degrees or even dual angles e.g. bevelled, may also be considered according to the intended operation. In this view, the loop 42 is minimised in size and the marker band 34 is visible, as are the cannula lumen 33 and the two cutting wire lumens 32. As can be seen, it is impossible for a cannula to interfere with the loop 42 of the cutting wire 40 in this mode, since the latter is fully retracted against the oblique distal end 31.

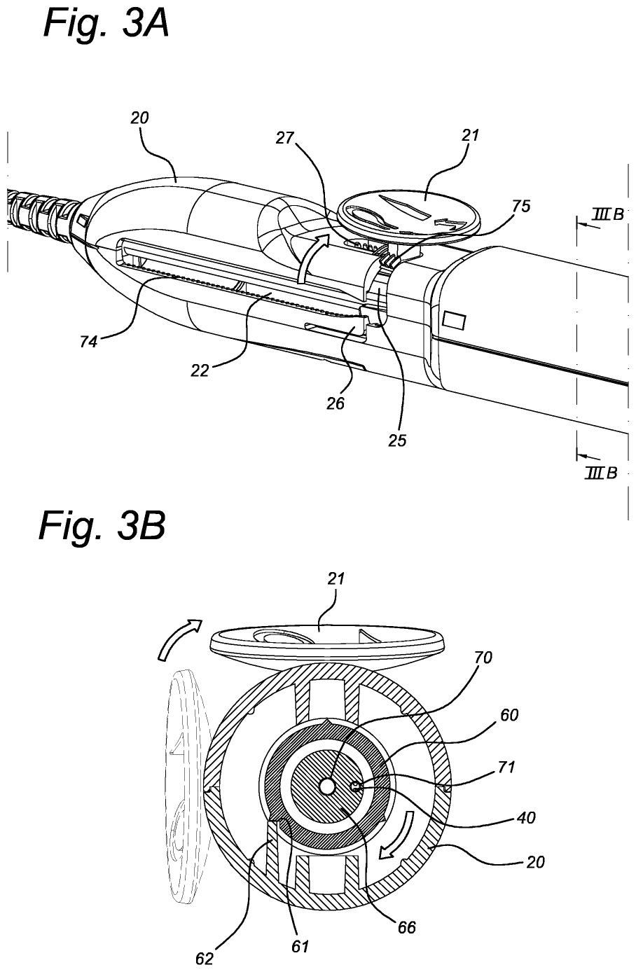

FIG. 3A schematically shows part of the catheter hub 20 after switching the slider 21 from cutting wire mode to cannula mode. The slider 21 has moved from the first slider channel 22 to the first position 24 in the connection channel 25 and subsequently through the connection channel 25 to a second slider channel 27. Both the first slider channel 22 and the second slider channel 27 are oriented to extend along a longitudinal axis defined by the shaft 30, preferably by a part of the shaft 30 that is closest to the catheter hub 20. The slider 21 can be moved from the first position 24 through the connection channel 25 over a circular arc of about 90 degrees centred around the longitudinal axis of the catheter hub 20. By moving the slider 21 through the connection channel 25 it stops engaging with the cutting wire 40 and instead engages with or grips the cannula 50, for instance by engaging with the cannula hub 52. The slider 21 can now move forward through the second slider channel 27 in order to move the cannula 50 through the shaft 30 to protrude from the shaft's distal end 31. Note that the second slider channel 27 is much shorter than the first slider channel 22 since the cannula 50 is configured to extend less far from the distal end 31 of the shaft 30 than the cutting wire 40.

The slider 21 and catheter hub 20 comprise several mechanisms to prevent the slider from sliding accidentally. In order to avoid protruding the cutting wire 40 after it has been fully retracted, the first slider channel 22 is provided with the safety detent 26 which blocks forward movement of the slider 21 when it is located in the first position 24 of the connection channel 25.

In addition, both the first slider channel 22 and second slider channel 27 are provided with a toothed rack 74. The slider 21 itself comprises teeth 75 also. The toothed rack 74 and teeth 75 are configured to engage with each other to block movement of the slider 21 in a direction along the first slider channel 22 or second slider channel 27 when the slider is 21 is not first depressed by a force towards the longitudinal axis by the user. Only when the user depresses the slider 21, the teeth 75 and toothed rack 74 will disengage, allowing the slider 21 to be moved through the first slider channel 22 or through the second slider channel 22.

FIG. 3B schematically shows a cross-section of the catheter assembly 1, through the catheter hub 20 in the direction III-B of FIG. 3A. The slider 21 is fixed to a hollow cylinder 60 which is rotatable about its own central axis. The central axis of the cylinder 60 coincides with the longitudinal axis of the shaft 30 and a central longitudinal axis of the catheter hub 20. The cylinder 60 comprises a groove 61 which is configured to engage with a ridge 62, which is connected to the interior of the catheter hub 20. When the slider 21 has been moved to cannula mode (in the direction indicated by the arrows), movement in the opposite direction is not possible anymore as this is blocked by the groove 61 and the ridge 62.

The cylinder 60 also comprises a centrally located channel separator 66 to be described further below, having a working channel 70 and a loop channel 71 passing therethrough. The elongate member 54 of a cannula 50 may be entered through the first end 28 of the catheter hub via the cannula carrier 29 and extends through the working channel 70 in the cylinder 60 into the cannula lumen 33 in the shaft. Both ends of the cutting wire 40 extend through the loop channel 71 as illustrated in FIG. 3B and from there connect to the cutting wire lumen 32.

The cylinder 60 is configured to engage with the cannula carrier 29 when being rotated, such that in cannula mode the cannula carrier 29 is translated along the longitudinal axis together with the slider 21. If a cannula hub 52 has been connected to the cannula carrier 29, the cannula carrier 29, cannula hub 52, and cannula 50 all move together with the slider 21. The cannula 50 moves through the working channel 70 and through the cannula lumen 33.

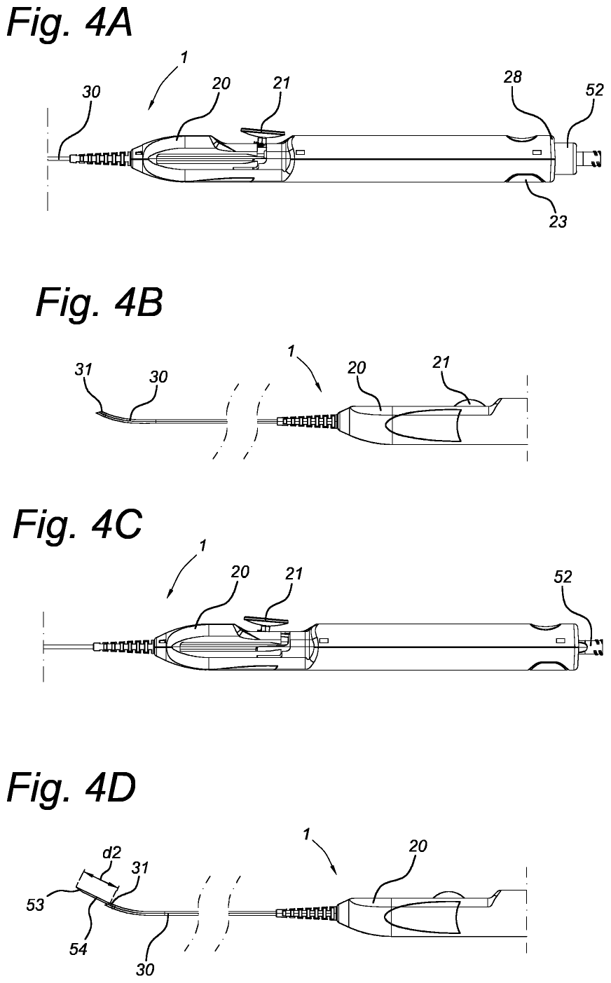

FIG. 4A shows what will be referred to as a side view of part of the catheter assembly 1 in cannula mode. FIG. 4B shows the catheter assembly of FIG. 4A rotated by 90 degrees to show a bottom view of the catheter hub 20 including the distal end 31 of the shaft 30. Parts of the catheter assembly 1 have been omitted, as indicated by the dotted and curved lines. At the first end 28 of the catheter hub 20, the cannula hub 52 as shown in FIG. 2A with cannula 50 has been arranged. The cannula 50 extends through the catheter hub 20 and through the working channel 70 into the cannula lumen 33 in the shaft 30. FIG. 4B shows that in the most backward position of the slider 21 as shown, the cannula 50 is completely retracted into the shaft 30, such that no part of the cannula 50 extends out of the shaft 30 at the distal end 31.

FIGS. 4C and 4D are corresponding views to FIGS. 4A and 4B but differ from FIGS. 4A-4B in that the slider 21 has moved forward through the second slider channel 27. The slider 21 operatively engages the cannula hub 52 such that the cannula 50 moves in the same direction. The cannula hub 52 has moved with the slider 21 at least partially into the catheter hub 20. The flexible elongate member 54 of the cannula 50 thus extends from the distal end 31 of the shaft 30. The flexible elongate member 54 of the cannula 50 extends from the distal end 31 for a second distance d2. The slider 21 can move back and forth through the second slider channel 27 to control the second distance d2. The second distance d2 may be maximised to between 5 and 20 mm, preferably about 10 mm. The flexible elongate member 54 of the cannula 50 may have a sharp point 53 to cut through a vessel's wall and/or calcified tissue to re-enter the vessel. In order to re-enter a vessel, the entire catheter assembly 1 is rotated such that the orientation of the bent end of the shaft 30 is directed towards the vessel before employing the cannula 50. In this way, the cannula 50 is able to cut through the vessel's wall and damage to other tissue is avoided.

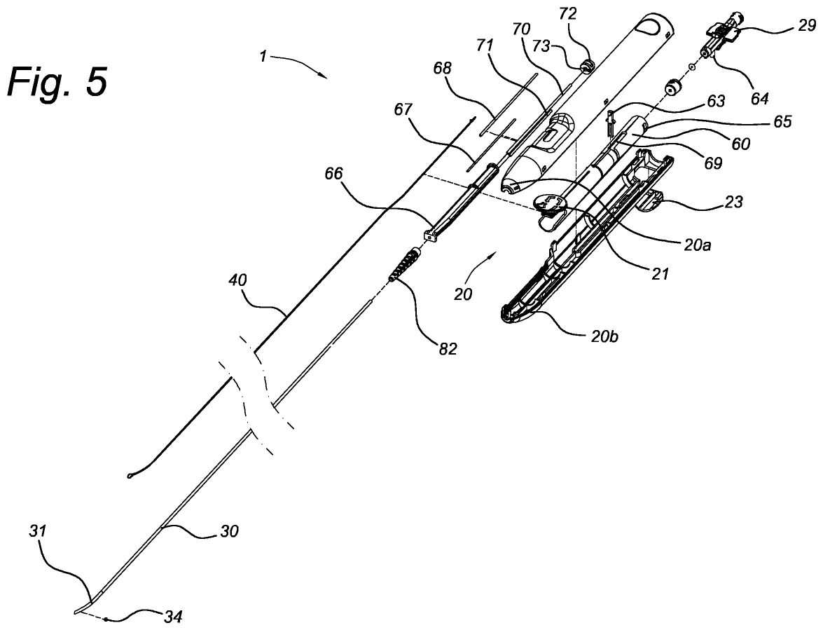

FIG. 5 schematically shows an exploded view of a catheter assembly 1, wherein part of the shaft 30 is omitted and wherein no cannula or guidewire is provided. The catheter hub 20 includes an upper shell 20a and a lower shell 20b between which, the hollow cylinder 60 as described in relation to FIG. 3B above is received. The slider 21 is connected to an outer surface of the cylinder 60. Inside the cylinder 60, the means for fixing and guiding the cannula 50 and cutting wire 40 are provided. In particular, the catheter assembly 1 comprises a channel separator 66 inside the cylinder 60. The channel separator 66 is fixed to the catheter hub 20 and does not move or rotate when the slider 21 and cylinder 60 are moved and/or rotated. The channel separator 66 separates the working channel 70 from the loop channel 71, directs the cutting wire 40 through the loop channel 71 into the cutting wire lumens 32, and directs the cannula 50 through the working channel 70 into the cannula lumen 33.

As explained above, the catheter assembly 1 can be used in a cutting wire mode or in a cannula mode wherein respectively the cutting wire 40 and the cannula 50 are allowed to operatively engage with the slider 21 while the other of the cutting wire 40 and cannula 50 is held fixed. Below the mechanism of engagement and disengagement of the cutting wire 40 and cannula 50 with the slider 21 are described.

In cutting wire mode, the first distance d1 that the cutting wire 40 extends from the distal end 31 can be manipulated by moving the slider 21. The cutting wire 40 is configured to extend between the catheter hub 20 and the distal end 31 of the shaft 30 through the loop channel 71 and the cutting wire lumens 32. The proximal ends of the cutting wire 40 are fixed within a cutting wire carrier inside the catheter hub 20. The cutting wire carrier comprises a loop fixation channel 67 and a loop fixation ring 72, which are together arranged to fix the ends of the cutting wire 40. The loop fixation ring 72 has a diameter smaller than an inner diameter of the hollow cylinder 60 and the loop fixation ring 72 may therefore move within the hollow cylinder 60.

A guide pin 63 controls the engagement of the loop fixation ring 72 with the hollow cylinder 60. As such, the guide pin 63 controls whether the cutting wire 40 moves along with the cylinder 60 when it is moved by the slider 21. In cutting wire mode, when the slider 21 moves through the first channel 33, the guide pin 63 engages the cutting wire carrier with the cylinder 60. Consequently, movement of the slider 21 and cylinder 60 along the longitudinal axis is translated into movement of the cutting wire carrier and into an increase or decrease of the first distance d1.

More precisely, the guide pin 63 controls the engagement of the loop fixation ring 72 to the cylinder 60. The cylinder 60 comprises an L-shaped groove 69 which is configured to accommodate the guide pin 63. In cutting wire mode, the guide pin 63 is blocked from any longitudinal movement, which also blocks the movement of the loop fixation ring 72 with respect to the cylinder 60. Consequently, when the slider 21 is moved along the first channel 21, the cylinder 60 and the loop fixation ring 72 are moved along.

In cannula mode, the loop fixation ring 72 is not blocked in its movement by the guide pin 63, as the guide pin 63 can move through a portion of the L-shaped groove 69 that extends along a direction parallel to the longitudinal axis. Therefore in cannula mode, the cylinder 60 can be moved along the longitudinal direction while the loop fixation ring 72 does not change its position. Consequently, in cannula mode, the position of the cutting wire carrier remains fixed with respect to the catheter hub 20 and shaft 30 and the cutting wire 40 is not extended but remains fully retracted.

The loop fixation ring 72 comprises a central opening 73 that allows the working channel 70 to extend through the loop fixation ring 72 and be moved independently of it.

In cutting wire mode, the cannula carrier 29 is not yet engaged with the cannula hub 52 and cannula 50. Only once a user has consciously decided to switch from cutting wire mode to cannula mode, can the cannula carrier 29 be engaged with the cylinder 60. To allow for such engagement, the user needs to deactivate the blocking mechanism that prevents the flexible elongate member 54 of the cannula 50 from extending while the cutting wire 40 is not fully retracted.

The cannula carrier 29 is configured to be engaged by the cylinder 60 by means of a connector pin 64 which can be accommodated in a second L-shaped groove 65. Such engagement may take place only after the slider 21 has been moved out of the first slider channel 22 and into the connection channel 25, i.e., when the slider 21 has been moved to the first position 24. When pressing the interlock button 23, the connector pin 64 can be moved into the corner of the second L-shaped groove 65, and only once the connector pin 64 is in that corner, the cannula carrier 29 can be rotated. Hence the interlock button 23 acts as a security mechanism that is configured to internally allow rotation of the cannula carrier 29 only when the interlock button 23 has been actuated. By actuating the interlock button 23 and simultaneously rotating the cannula carrier 29, the cannula carrier 29 engages with the cylinder 60 and the blocking mechanism configured to block movement of the cannula carrier 29 disengages. The slider 21 may now be moved through the second channel 22 to manipulate the cannula 50 to extend from the distal end 31. As explained above, when the slider 21 is moved through the second channel 22, the pin 63 does not engage and therefore the cutting wire 40 remains retracted.

Before engaging the cannula carrier 29 with the cylinder 60, a cannula 50 can already be inserted through the cannula carrier 29 and interlocked with the cannula carrier 29 via the cannula hub 52. Preferably, however, the cannula hub 52 is only interlocked with the cannula carrier 29 when the catheter assembly 1 is switched to cannula mode. The cannula carrier 29 comprises a connector 68, which guides the cannula 50 into the working channel 70. The connector 68 is fixed inside the cannula carrier 29, and has a smaller outer diameter than the inner diameter of the working channel cannula 70. This allows the connector cannula 68 to move inside the working channel cannula 70. This configuration allows for a leakage proof connection.

The invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

1. A catheter assembly for passing an occlusion in a vessel, comprising:
a catheter hub;
a hollow flexible shaft extending between the catheter hub and a distal end, wherein the shaft comprises a cannula lumen with a cannula opening at the distal end, the cannula lumen being configured to guide a cannula, and at least one cutting wire lumen;
a cutting wire provided inside the shaft, the cutting wire extending through the cutting wire lumen from a cutting wire carrier within the catheter hub to a cutting wire opening at the distal end;
a cannula carrier, provided within the catheter hub, the cannula carrier being configured to receive a cannula and being aligned with the cannula lumen whereby a cannula can pass through the cannula carrier and through the cannula lumen;
wherein the cannula carrier and the cutting wire carrier are slideably received within the catheter hub to allow extension and retraction of the respective cannula and cutting wire and the catheter hub is configured to operate selectively in a cutting wire mode and in a cannula mode,
wherein
in cutting wire mode a user can act on the cutting wire carrier to move the cutting wire to extend from the cutting wire opening and to be retracted into the shaft;
in cannula mode the user can act on the cannula carrier to move the cannula to extend from the cannula opening; and
wherein the catheter hub further comprises a blocking mechanism, configured to block movement of the cannula carrier while the cutting wire extends from the cutting wire opening.

2. The catheter assembly according to claim 1, wherein the catheter hub includes a handle and the handle is configured to operate selectively in a cutting wire mode and in a cannula mode,
wherein
in cutting wire mode the handle operatively engages with the cutting wire carrier such that a user can act on the handle to move the cutting wire to extend from the cutting wire opening and to be retracted into the shaft;
in cannula mode the handle operatively engages with the cannula carrier such that a user can act on the handle to move the cannula to extend from the cannula opening.

3. The catheter assembly according to claim 2, wherein the handle is a slider and wherein the catheter hub comprises a first slider channel and a second slider channel, which are preferably both oriented in a direction defined by a longitudinal axis of the shaft, wherein the slider is configured to slide through the first slider channel to control the advancement of the cutting wire in cutting wire mode;
wherein the slider is configured to slide through the second slider channel to control the advancement of the cannula in cannula mode; and
wherein the first slider channel and second slider channel are connected such that the slider can move from the first slider channel to the second slider channel.

4. The catheter assembly according to claim 3, wherein the first slider channel and/or the second slider channel is provided with a toothed rack and the slider has at least one tooth for engaging with the toothed rack to block movement in a direction along the first or second slider channel, such that the slider can only slide through the first or second slider channel when the slider is depressed by a user to disengage the tooth and the rack.

5. The catheter assembly according to claim 2, wherein a first position of the handle corresponds to a fully retracted cutting wire, and wherein the blocking mechanism comprises a safety detent configured to fix the handle at the first position when it reaches this position, such that the handle is blocked from advancing the cutting wire.

6. The catheter assembly according to claim 1, wherein the catheter hub includes a first handle and a second handle, wherein
the first handle is connected to or configured to operatively engage with the cutting wire carrier such that a user can act on the first handle to move the cutting wire to extend outside from the cutting wire opening and to be retracted into the shaft; and
the second handle is connected to or configured to operatively engage with the cannula carrier such that a user can act on the second handle to move the cannula to extend from the cannula opening.

7. The catheter assembly according to claim 2, wherein the catheter hub is elongate and extends between a first end and an opposite second end, which is connected to the shaft, wherein the catheter hub comprises a grip for gripping the catheter hub by a user, and wherein the handle is configured to move in a handle region which is between the grip and the second end, such that the movement of the handle can be controlled by a user's thumb.

8. (canceled)

9. (canceled)

10. The catheter assembly according to claim 1, wherein the catheter hub comprises an interlock button, preferably near a proximal end of the catheter hub, and wherein full retraction of the cutting wire into the shaft is only possible while the interlock button is actuated.

11. The catheter assembly according to claim 1, wherein the cannula carrier or a proximal end of the catheter hub is configured to lockingly engage a cannula hub, whereby once engaged, the cannula hub cannot be separated from the cannula carrier or catheter hub, and wherein the cannula hub is configured to be fixed to the cannula carrier or catheter hub, preferably when the handle switches to cannula mode.

12. The catheter assembly according to claim 1, wherein the cutting wire comprises a loop at its distal end.

13. The catheter assembly according to claim 12, wherein the cutting wire includes two parallel cutting wire portions extending from the catheter hub to the distal end, which are continuous with one another at the distal end, so as to form the loop.

14. The catheter assembly according to claim 13, wherein the two cutting wire portions are inside separate cutting wire lumens, extending from cutting wire openings at the distal end to form the loop.

15. The catheter assembly according to claim 12, wherein one end of the cutting wire is engaged with the cutting wire carrier and the other end of the cutting wire is fixed, preferably to the shaft or the catheter hub.

16. (canceled)

17. (canceled)

18. (canceled)

19. (canceled)

20. A cannula for use in the catheter assembly according to claim 1, wherein the cannula comprises an elongate, flexible member and a cannula hub for fixing the cannula to the catheter hub or cannula carrier, the cannula hub being connected to one end of the flexible member.

21. The cannula according to claim 20, wherein the flexible member is made of PEEK.

22. A method for operating the catheter assembly according to claim 1, the method comprising:
extending the cutting wire from the distal end;
fully retracting the cutting wire into or against the shaft; and subsequently
extending a cannula from the cannula opening, whereby the cutting wire is precluded from interfering with the cannula.

23. The method according to claim 22, further comprising
positioning a cannula within the cannula lumen, preferably prior to extending the cutting wire.

24. The method according to claim 22, wherein the method is used for passing an occlusion in a vessel, and further comprises:
introducing the shaft of the catheter assembly into the vessel;
wherein extending the cutting wire includes cutting through at least one layer of the vessel wall, in order to pass the occlusion; and
wherein extending the cannula includes cutting through the tissue, in order to re-enter the vessel.

25. The method according to claim 22, further comprising:
switching from cutting wire mode to cannula mode after retracting the cutting wire.

26. The method according to claim 22, further comprising:
inserting a guide wire into and through the cannula to extend from the cannula distal end, distal of the occlusion; and
retracting the catheter and cannula from the vessel while leaving the guide wire in position, across the occlusion.
